(19) United States
(12) Patent Application Publication
(10) Pub. No.: US 2026/0157637 A1
(43) Pub. Date: Jun. 11, 2026

(54) METHOD OF OPERATION OF AN APPARATUS FOR PREDICTING DISEASE BASED ON BODY TEMPERATURE DATA MEASURED IN A NON-CONTACT MANNER

(71) Applicant: Meta Therapeutics Inc., Gwangju (KR)

(72) Inventor: Jangwon LEE, Seoul (KR)

(73) Assignee: Meta Therapeutics Inc., Gwangju (KR)

(21) Appl. No.: 19/361,583

(22) Filed: Oct. 17, 2025

(30) Foreign Application Priority Data
Dec. 10, 2024 (KR) 10-2024-0182949

(51) Int. Cl. A61B 5/01; A61B 5/00; G16H 10/20

(52) U.S. Cl. CPC A61B 5/01; A61B 5/7246; A61B 5/7267; A61B 5/7275; G16H 10/20

(57) ABSTRACT

The present disclosure relates to a method of operating an electronic device connected to a user terminal and a non-contact body temperature measurement device. The operating method includes acquiring variable data including a user’s age and sex through user input received from the user terminal; setting a user’s normal body temperature range based on data output by inputting the variable data including the user’s age and sex to at least one artificial intelligence model trained to determine the normal body temperature range based on at least one element included in the variable data; and comparing the user’s body temperature sensed by the body temperature measurement device with the set normal body temperature range to predict the user’s disease.

### CROSS-REFERENCE TO RELATED APPLICATION

This application claims the benefit of and priority to Korean Patent Application No. 10-2024-0182949, filed on December 10,2024, the entire disclosure(s) of which is hereby incorporated herein by reference in its entirety.

### BACKGROUND

### Field

The present disclosure relates to a method of operating an electronic device connected to a user terminal and a non-contact body temperature measurement device, and more specifically, to a method of operating an electronic device that predicts a user's disease by setting a user-customized normal body temperature range based on user data and identifying a fever pattern based on a user’s body temperature measured by a non-contact body temperature measurement device and the user-customized normal body temperature range.

### Description of Related Art

Wearable devices are computers implemented in a wearable form and are being developed and released in various forms such as smartwatches, rings, and glasses. Users can be linked to user devices such as smartphones through the wearable devices to receive notifications and monitor activity and physical data of the users, such as a heart rate, the number of steps, and electrocardiograms, thereby easily finding a variety of applications that utilize the data.

The accuracy of the data utilized by the applications needs to be supported to improve the accuracy of information obtained through these applications. However, a measurement environment for biometric data using the wearable device varies from user to user, which may lead to reduced accuracy of the biometric data measured by the wearable device.

It should be considered that, for a body temperature in the biometric data, a normal body temperature range varies depending on various variables, such as an age, body temperature measurement site, body temperature measurement point in time, and sex. In order to ensure the accuracy of identification of whether a fever occurs, it is necessary to identify whether the fever occurs based on an appropriate body temperature range, and thus, there is a need for a technology for setting a user-customized normal body temperature range, in consideration of various variables.

### Prior Art Document

### Patent Document

Patent Document 1 Korean Patent Publication No. 10-2320224

### SUMMARY

The present disclosure is intended to provide a method of operating an electronic device that predicts a user's disease by setting a user-customized normal body temperature range based on user data and identifying a fever pattern based on a user’s body temperature measured by a non-contact body temperature measurement device and the user-customized normal body temperature range.

The objects of the present disclosure are not limited to the aforementioned object, and other objects and advantages of the present disclosure that are not mentioned may be understood through the following description and will be more clearly understood by the embodiments of the present disclosure. Further, it will be easily understood that the objects and advantages of the present disclosure can be realized by the means and combinations thereof set forth in the claims.

According to an embodiment of the present disclosure, a method of operating an electronic device connected to a user terminal and a non-contact body temperature measurement device includes acquiring variable data including a user’s age and sex through user input received from the user terminal; setting a user’s normal body temperature range based on data output by inputting the variable data including the user’s age and sex to at least one artificial intelligence model trained to determine the normal body temperature range based on at least one element included in the variable data; and comparing the user’s body temperature sensed by the body temperature measurement device with the set normal body temperature range to predict the user’s disease.

In this case, the method of operating an electronic device may include receiving information on an external environment temperature of the body temperature measurement device from the body temperature measurement device; acquiring information on at least one physical activity performed by the user through the user input received from the user terminal; and adding information on the external environment temperature and the body activity to the variable data.

Meanwhile, the method of operating an electronic device may include acquiring training data including a plurality of body temperature ranges and at least one variable element matching each of the plurality of body temperature ranges; and training the artificial intelligence model based on the training data.

Meanwhile, the predicting of the user’s disease may include identifying the user’s fever pattern including the user’s hourly body temperature deviating from the set normal body temperature range when the user’s body temperature deviates from the set normal body temperature range; comparing the user’s fever pattern with the fever pattern preset for each disease to acquire a similarity; and identifying a disease for which the similarity exceeds a threshold as the user’s disease.

Here, the identifying of the user’s fever pattern may include identifying at least one fever point in time at which the user’s body temperature begins to deviate from the set normal body temperature range; calculating, for each fever point in time, a fever period during which the user’s body temperature continuously deviates from the set normal body temperature range; identifying, for each fever point in time, whether the user’s body temperature increases or decreases; and identifying the fever pattern including at least one of a time interval between a plurality of fever points in time, a fever period for each of the plurality of fever points in time, and whether the user’s body temperature increases or decreases.

In this case, the comparing of the user’s fever pattern with the fever pattern preset for each disease to acquire the similarity may include acquiring the similarity of the user’s fever pattern to the fever pattern preset for each disease based on data output by inputting the user’s fever pattern and the fever pattern preset for each disease to at least one similarity calculation model trained to calculate similarity between data based on data embedding.

Meanwhile, the method of operating an electronic device may include identifying a body temperature pattern related to the user’s body temperature change cycle, based on a body temperature measurement history including the user’s hourly body temperature divided by day; and correcting the user’s normal body temperature range based on the identified body temperature pattern.

Here, the identifying of the body temperature pattern may include identifying at least one rising time period in which the user’s body temperature rises daily and identifying at least one first time period in which a plurality of ones of the daily identified rising time periods overlap; identifying at least one falling time period in which the user’s body temperature falls daily and identifying at least one second time period in which a plurality of ones of the daily identified falling time periods overlap; and identifying the user’s body temperature pattern including the first time period and the second time period.

In this case, the correcting of the user’s normal body temperature range may include calculating a first average value, which is an average value of the body temperature identified during the first time period, and a second average value, which is an average value of body temperature identified during the second time period, based on the body temperature measurement history; correcting the user’s normal body temperature range based on the first average value to acquire a first body temperature range; setting the first body temperature range to the user’s normal body temperature range during the first time period; correcting the user’s normal body temperature range based on the second average value to acquire a second body temperature range; and setting the second body temperature range as the user’s normal body temperature range during the second time period.

According to the present disclosure, it is possible to improve the accuracy of body temperature monitoring by determining whether a fever is present based on a normal body temperature range corrected by reflecting various user variables, such as age, sex, and body temperature measurement site.

According to the present disclosure, it is also possible to provide information on a disease predicted to have occurred to the user based on the user’s fever pattern, thereby encouraging the user to recognize the severity of the fever and visit a hospital.

### BRIEF DESCRIPTION OF THE DRAWING

FIG. 1 is a flowchart illustrating an operation in which an electronic device 100 predicts a user's disease according to an embodiment of the present disclosure.

FIG. 2 is a diagram illustrating an operation in which the electronic device performs communication with various devices according to an embodiment of the present disclosure.

FIG. 3 is a flowchart illustrating an operation in which the electronic device predicts the user’s disease based on the user’s fever pattern according to an embodiment of the present disclosure.

FIG. 4 is a flowchart illustrating an operation in which the electronic device corrects a normal body temperature range based on the user’s body temperature pattern according to an embodiment of the present disclosure.

FIG. 5 is a block diagram illustrating a configuration of the electronic device according to the embodiment of the present disclosure.

### DETAILED DESCRIPTION

A method of describing the present specification and drawings will be described before the present disclosure is described in detail.

First, the terms used in the present specification and claims are general terms selected in consideration of functions of various embodiments of the present disclosure. However, these terms may vary depending on the intentions of those skilled in the art, legal or technical interpretation, and the emergence of new technologies. Further, some terms are arbitrarily selected by the applicant. These terms may be construed based on the meanings defined in the present specification, and when a term is not specifically defined, the term may be constructed based on the overall content of the present specification and common technical knowledge in the relevant technical field.

Further, the same reference numbers or symbols in the drawings appended to the present specification indicate parts or components that perform substantially the same functions. For convenience of explanation and understanding, the same reference numbers or symbols are used in different embodiments. In other words, even when components with the same reference number are depicted in a plurality of drawings, the plurality of drawings do not represent a single embodiment.

Further, terms including ordinal numbers, such as “first,” “second,” may be used in the present specification and claims to distinguish between components. These ordinal numbers are used to distinguish the same or similar components from each other, and meanings of the terms should not be construed as limited by use of the ordinal numbers. For example, components associated with the ordinal numbers should not be restricted in an order of use or arrangement by their numbers. When necessary, each ordinal number may be used interchangeably.

In the present specification, singular expressions include plural expressions unless the context clearly indicates otherwise. In the present application, terms such as “comprise” or “configured” are intended to indicate the presence of features, numbers, steps, operations, components, parts, or combinations thereof described in the specification, but should be understood not to preemptively exclude the possibility of the presence or addition of one or more other features, numbers, steps, operations, components, parts, or combinations thereof.

In the embodiments of the present disclosure, terms such as “module,” “unit,” and “part” refer to a component that performs at least one function or operation, and the components may be implemented in hardware, software, or a combination of hardware and software. Further, a plurality of “modules,” “units,” “parts,” and the like may be integrated into at least one module or chip and implemented as at least one processor, except for a case in which the modules, units, and parts need to be implemented as individual specific hardware.

Further, in the embodiments of the present disclosure, when a part is said to be connected to another part, this includes not only a direct connection but also an indirect connection via another medium. Furthermore, unless specifically stated otherwise, a part including a component does not exclude inclusion of other components but rather implies the inclusion of the other components.

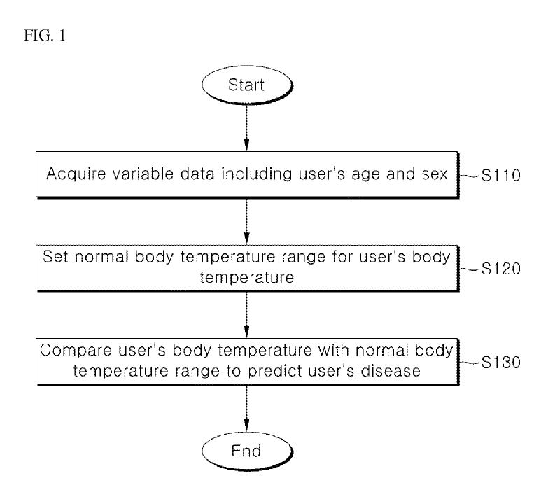

FIG. 1 is a flowchart illustrating an operation in which an electronic device 100 predicts a user's disease according to an embodiment of the present disclosure.

The electronic device 100 may provide a service for predicting a user's disease based on a user’s body temperature.

In an embodiment, the electronic device 100 may be implemented as a server including one or more computers. In this case, the electronic device 100 may be linked with a user terminal 200 through at least one application or web page to provide the above-described service to the user. Further, the electronic device 100 may provide software (computer program) to at least one user terminal, and may also provide the above-described service to the user terminal 200 that has executed the software.

As an additional embodiment, the electronic device 100 may correspond to a user terminal in which at least one application or software (computer program) providing the above-described service is installed. In this case, the electronic device 100 may correspond to a user terminal such as a desktop PC, a tablet PC, a laptop PC, a smartphone, or a wearable device, or may be implemented as a kiosk installed, a public PC, or the like at a specific place.

In addition, the electronic device 100 may be implemented as various servers or terminal devices.

Referring to FIG. 1, the electronic device 100 may be connected to the user terminal 200 to acquire variable data including the user’s age and sex through user input received from the user terminal (S110).

The user terminal 200 may correspond to a user terminal device implemented as a smartphone, a wearable device, a tablet PC, a laptop PC, a desktop PC, or the like.

In addition, the electronic device 100 may be connected to a body temperature measurement device 300, which measures a temperature in a non-contact manner, to receive information on an external environment temperature of the body temperature measurement device 300.

For example, the body temperature measurement device 300 may be a device that measures a temperature in a non-contact manner that detects infrared radiation emitted from skin using an infrared temperature sensor.

Accordingly, the electronic device 100 may receive the external environment temperature of the body temperature measurement device 300 or the user’s body temperature from the body temperature measurement device 300.

In addition, the electronic device 100 may acquire information on at least one physical activity (for example, running, walking, or cycling) performed by the user through user input received from the user terminal 200.

Accordingly, the electronic device 100 may add information on the external environment temperature and the information on the physical activity to the variable data.

The electronic device 100 may set a normal body temperature range for the user’s body temperature based on the variable data (S120).

In an embodiment, the electronic device 100 may include at least one artificial intelligence model trained to set the normal body temperature range based on at least one element (for example, sex, age, external environment temperature, and performed physical activity) included in the variable data.

The artificial intelligence model may be a model trained based on training data including a plurality of body temperature ranges and at least one element (for example, age range, sex, environment temperature, or physical activity performed) matched with each of the plurality of body temperature ranges, or may be a model based on various learning algorithms such as a recurrent neural network (RNN), a long short-term memory (LSTM), a random forest, or a decision tree, but not limited thereto.

Accordingly, the electronic device 100 may set the user’s normal body temperature range based on data output by inputting the variable data to the artificial intelligence model.

In a further embodiment, the electronic device 100 may set a body temperature range corresponding to the user's sex and age included in the variable data among a plurality of body temperature ranges previously stored to be matched to a plurality of age groups and sexes, as a user's reference body temperature range.

In this case, the electronic device 100 may acquire the user’s normal body temperature range by correcting the reference body temperature range based on the information on the external environment temperature and the activity performed by the user.

Specifically, the electronic device 100 may set an environmental correction coefficient based on a difference between the external environment temperature included in the variable data and a preset reference environment temperature, and identify an activity correction coefficient pre-matched with the physical activity performed by the user.

Accordingly, the electronic device 100 may acquire the user’s normal body temperature range by applying the environmental correction coefficient and the activity correction coefficient to the reference body temperature range.

Here, when the user is identified as having performed a plurality of physical activities, the electronic device 100 may acquire the normal body temperature range by applying, to the reference body temperature range, a coefficient calculated by applying the number of physical activities performed by the user to an activity correction coefficient pre-matched with a physical activity performed most recently by the user.

The electronic device 100 may compare the user’s body temperature sensed by the body temperature measurement device 300 with the user’s normal body temperature range to predict the user’s disease (S130).

In an embodiment, the electronic device 100 may identify that the user is in a fever state when the user’s body temperature sensed through the body temperature measurement device 300 exceeds the normal body temperature range.

In this case, the electronic device 100 may identify a fever pattern including the user’s hourly body temperature and predict the user’s disease based on the identified fever pattern.

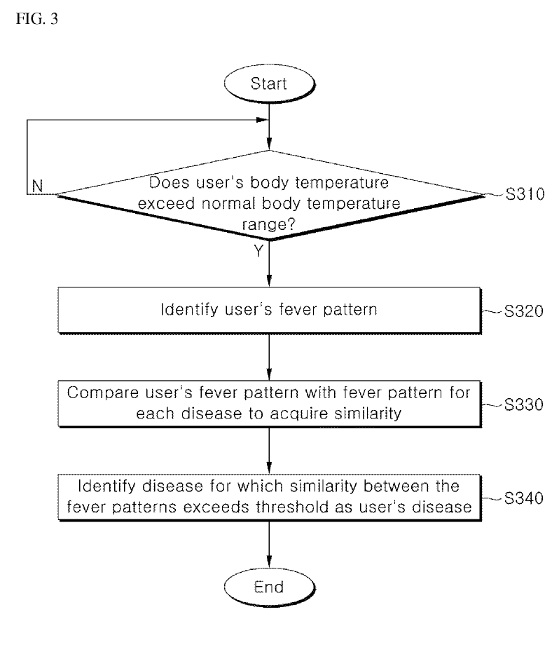

A more detailed description of this will be given below with reference to FIG. 3.

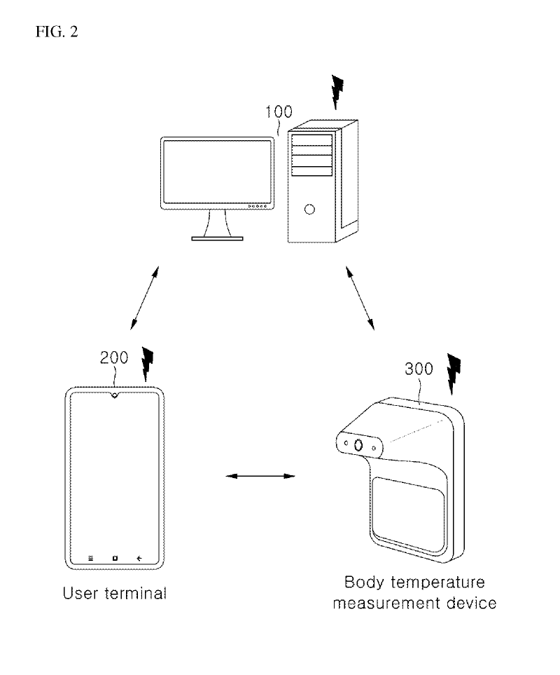

FIG. 2 is a diagram illustrating an operation in which the electronic device performs communication with various devices according to an embodiment of the present disclosure.

Referring to FIG. 2, the electronic device 100 may be connected to and communicate with the user terminal 200 and the body temperature measurement device 300.

Here, the body temperature measurement device 300 may correspond to a device connected to the user terminal 200.

Accordingly, the electronic device 100 may receive identification information of the body temperature measurement device 300 connected to the user terminal 200 from the user terminal 200, and identify the body temperature sensed by the body temperature measurement device 300 corresponding to the received identification information as the body temperature of the user of the user terminal 200.

Meanwhile, the electronic device 100 may provide a first user Interface (UI) for inputting variable data, including the user's sex, age, physical activity, and the like to the user terminal 200 through at least one application or web page.

In addition, the electronic device 100 may provide a second UI for providing information on the user’s disease predicted based on the user’s body temperature sensed by the body temperature measurement device 300 and the user’s normal body temperature range set based on the variable data.

In this case, the electronic device 100 may provide the user terminal 200 with a third UI including a first UI item for inputting information on whether the user’s disease is true (whether the user’s actual disease matches the user’s disease provided through the second UI).

Here, when the user’s actual disease is identified as not matching the user’s disease provided through the second UI based on user input that has been input through the third UI, the electronic device 100 may provide the user terminal 200 with a fourth UI including a second UI item for inputting information on the user’s actual disease.

Accordingly, the electronic device 100 may identify a similarity between a fever pattern preset for the user’s disease (predicted disease) provided through the second UI and a fever pattern preset for a disease based on the information on the user’s actual disease acquired through the fourth UI (actual disease).

Here, when the identified similarity exceeds a threshold, the electronic device 100 may provide the user with both information on the predicted disease and information on the actual disease if the same disease as the predicted disease is predicted as the user's disease or the same disease as the actual disease is predicted as the user's disease, based on the user's fever pattern that is subsequently identified.

FIG. 3 is a flowchart illustrating an operation in which the electronic device predicts the user’s disease based on the user’s fever pattern according to an embodiment of the present disclosure.

Referring to FIG. 3, the electronic device 100 may identify whether the user’s body temperature sensed by the body temperature measurement device 300 exceeds the normal body temperature range (S310).

Accordingly, when the user’s body temperature falls within the normal body temperature range, the electronic device 100 may perform step S310 again if the user’s body temperature sensed by the body temperature measurement device 300 is received.

When the user’s body temperature exceeds the normal body temperature range, the electronic device 100 may identify the user's fever pattern (S320).

In an embodiment, the electronic device 100 may identify the fever pattern including the user’s hourly body temperature deviating from the normal body temperature range.

Specifically, the electronic device 100 may set, as a fever point in time, a point in time when the user’s body temperature begins to deviate from the normal body temperature range set for the user.

When the fever point in time is identified, the electronic device 100 may calculate a time when the user’s body temperature is sensed to continuously deviate from the normal body temperature range through the body temperature measurement device 300, from the fever point in time, and set the fever period.

Furthermore, when a plurality of fever points in time are identified, the electronic device 100 may calculate a time when the user’s body temperature is sensed to continuously deviate from the normal body temperature range through the body temperature measurement device 300, from each of the plurality of fever points in time, and set a fever period for each of the plurality of fever points in time.

In addition, the electronic device 100 may identify, for each fever point in time, whether the user’s body temperature increases or decreases in the fever period when the user’s body temperature is identified as exceeding the normal body temperature range.

Accordingly, the electronic device 100 may identify a fever pattern including at least one of a time interval between the plurality of fever points in time, the fever period calculated for each fever point in time, and whether the user’s body temperature increases or decreases as identified for each fever point in time.

The electronic device 100 may compare the user’s fever pattern with a fever pattern for each disease to acquire a similarity (S330).

For example, the diseases may include colds, pneumonia, COVID-19, typhoid fever, meningitis, bacterial diseases, malaria, tuberculosis, and the like, and the fever pattern may include, but are not limited to, information on repeated increases and decreases in body temperature, a graph of a gradually increasing body temperature, information on a duration of a body temperature exceeding the normal body temperature range, a fever point in time identified during a specific time period (for example, morning, afternoon, or 8 PM to 3 AM), and the like.

Specifically, the electronic device 100 may include at least one similarity calculation model trained to calculate similarity between data.

For example, the electronic device 100 may include a first similarity calculation model including a first deep learning model trained to convert text into vectors and calculate similarity based on a distance between the vectors. In this case, the first deep learning model may include at least one embedding module for converting text constituting each piece of information into a vector space. Further, the first similarity calculation model may be implemented based on an algorithm such as an SVM or a random forest. In this case, the distance between the vectors may be calculated based on a difference in vector values for dimensions, and the first similarity calculation model may be a model trained to calculate higher similarity as the distance between vectors decreases.

In this case, the electronic device 100 may acquire the similarity between the user’s fever pattern and a fever pattern preset for each disease based on data output by inputting the user’s fever pattern and the fever pattern preset for each disease to the first similarity calculation model.

As an additional example, the electronic device 100 may generate a fever pattern in which the user’s body temperature sensed at each of a plurality of points in time through the body temperature measurement device 300 is implemented in a graph format (for example, a time-body temperature graph).

In this case, the electronic device 100 may include a second similarity calculation model including a second deep learning model trained to calculate similarity between graphs by performing embedding, which converts a graph into a vector reflecting graph characteristics such as nodes and edges within the graph. In this case, the second deep learning model may include at least one embedding module for converting a graph constituting each piece of information into a vector space. In this case, the second similarity calculation model may correspond to a model trained based on various learning algorithms such as Graph Convolutional Networks (GCN), Graph Neural Networks (GNN), Graph Attention Networks (GAT), and Graph Matching Networks (GMN) , but not limited thereto. In this case, the distance between the vectors may be calculated based on a difference between vector values for each dimension, and the second similarity calculation model may be a model trained to calculate a higher similarity as the distance between the vectors decreases.

Accordingly, the electronic device 100 may acquire the similarity between the user’s fever pattern and the fever pattern preset for each disease based on the data output by inputting the user’s fever pattern and the fever pattern preset for each disease to the second similarity calculation model.

The electronic device 100 may identify a disease for which the similarity between the fever patterns exceeds a threshold as the user’s disease (S340).

In this case, the electronic device 100 may transmit disease information (for example, disease name, symptoms, related hospital type (for example, internal medicine, otolaryngology, or dermatology)) pre-stored to be matched with a disease identified as the user’s disease to the user terminal 200.

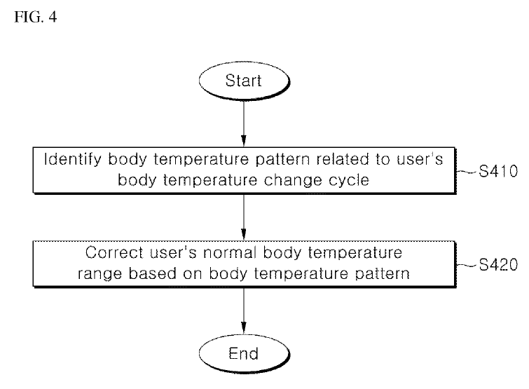

FIG. 4 is a flowchart illustrating an operation in which the electronic device corrects the normal body temperature range based on the user’s body temperature pattern according to an embodiment of the present disclosure.

Referring to FIG. 4, the electronic device 100 may identify a body temperature pattern related to the user’s body temperature change cycle, based on a body temperature measurement history including the user’s hourly body temperature divided by day (S410).

Specifically, the electronic device 100 may identify, for each day, at least one rising time period (for example, 12:00 to 1:00 or 4:00 to 6:00) in which the user’s body temperature rises from the body temperature measurement history but does not exceed the normal body temperature range, and may identify at least one first time period in which a plurality of ones of the rising time periods overlap.

For example, when a rising time period of 12:00 to 1:00 is identified on a first day, 11:00 to 12:30 on a second day, and 12:00 to 12:40 on a third day, the electronic device 100 may identify 12:00 to 12:30 as the first time period.

Further, the electronic device 100 may identify, for each day, at least one falling time period in which the user’s body temperature falls from the body temperature measurement history, and may identify at least one second time period in which a plurality of ones of the falling time periods overlap.

This makes it possible for the electronic device 100 to identify a body temperature pattern including the first time period and the second time period.

The electronic device 100 may correct the user’s normal body temperature range based on the body temperature pattern (S420).

Specifically, the electronic device 100 may calculate a first average value, which is an average value of the body temperature identified during the first time period, based on the body temperature measurement history, and a second average value, which is an average value of the body temperature identified during the second time period.

Therefore, the electronic device 100 may correct the normal body temperature range set for the user’s body temperature based on the first average value, to acquire a first normal body temperature range.

Specifically, the electronic device 100 may identify upper and lower limits of the normal body temperature range set for the user’s body temperature, set the lower limit as a lower limit of the first normal body temperature range, and set a value obtained by adding a difference between the lower limit and the first average value to the first average value as an upper limit of the first normal body temperature range. Thus, a median value of the first normal body temperature range may be set as the first average value.

For example, when the normal body temperature range set for the user’s body temperature is from 36°C to 37°C and the first average value is calculated as 36.7°C, the electronic device 100 may calculate a difference between 36°C, which is a lower limit of the normal body temperature range, and the first average value as 0.7, thereby setting the first normal body temperature range as 36°C to 37.4°C.

In addition, the electronic device 100 may correct the normal body temperature range set for the user’s body temperature based on the second average value to acquire a second normal body temperature range.

For example, the electronic device 100 may identify the lower limit of the normal body temperature range set for the user’s body temperature, set the upper limit as an upper limit of the second normal body temperature range, and set a value obtained by subtracting a difference between the upper limit and the second average from the second average as a lower limit of the second normal body temperature range. Thus, a median value of the second normal body temperature range may be set as the second average.

For example, when the normal body temperature range set for the user’s body temperature is from 36°C to 37°C and the second average value is calculated as 36.4°C, the electronic device 100 may calculate a difference between 37°C, which is the upper limit of the normal body temperature range, and the second average value as 0.6, thereby setting the second normal body temperature range as 35.7°C to 37°C.

In this case, the electronic device 100 may set the first normal body temperature range as the normal body temperature range for the user’s body temperature during the first time period, and set the second normal body temperature range as the normal body temperature range for the user’s body temperature during the second time period.

This makes it possible for the electronic device 100 to predict the user’s disease based on more precise fever data by applying different normal body temperature ranges to a time period in which the user’s body temperature is measured as a high temperature and a time period in which the body temperature is measured as a low temperature, depending on the user's lifestyle pattern or lifestyle environment.

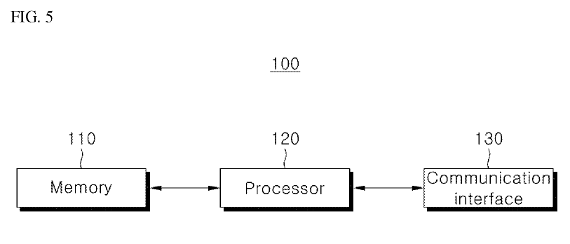

FIG. 5 is a block diagram illustrating a configuration of the electronic device according to the embodiment of the present disclosure.

Referring to FIG. 5, the electronic device 100 may include a memory 110, a processor 120, and a communication interface 130.

The memory 110 is a component for storing an operating system (OS) for controlling the overall operation of the components of the electronic device 100, and at least one instruction or data related to the components of the electronic device 100.

The memory 110 may include a non-volatile memory such as a ROM and a flash memory, and may include a volatile memory including a DRAM. In addition, the memory 110 may include a hard disk, a solid state drive (SSD), and the like.

The processor 120 is a component for overall control of the electronic device 100.

In an embodiment, the processor 120 may include, for example, a general-purpose processor such as a central processing unit (CPU), an AP, or a digital signal processor (DSP), a graphics-only processor such as a graphics processor unit (GPU) or a vision processing unit (VPU), or an AI-only processor such as a neural processing unit (NPU). The AI-only processor may be designed with a hardware structure specialized for training or using of a specific AI model.

The communication interface 130 is a component for performing communication with the outside.

The communication interface 130 may include a circuit, a module, a chip, and the like for performing communication using various wired and wireless communication schemes. The communication interface 130 may also be connected to an external device and a server via various networks.

The network may be a personal area network (PAN), a local area network (LAN), a wide area network (WAN), or the like depending on an area or scale, and may be an Intranet, an Extranet, the Internet, or the like depending on openness of the network.

The communication interface 130 may be connected to an external device and a server via various wireless communication schemes, such as long-term evolution (LTE), LTE Advance (LTE-A), 5th Generation (5G) mobile communication, code division multiple access (CDMA), wideband CDMA (WCDMA), universal mobile telecommunications system (UMTS), wireless broadband (WiBro), global system for mobile communications (GSM), time division multiple access (DMA), WiFi (Wi-Fi), WiFi Direct, Bluetooth, Bluetooth Low Energy (BLE), near field communication (NFC), Zigbee, and LoRa.

Further, the communication interface 130 may be connected to an external device and a server via wired communication schemes such as Ethernet, an optical network, universal serial bus (USB), or Thunderbolt.

Furthermore, the communication interface 130 may be configured to utilize various new communication schemes/technologies developed in the future.

Meanwhile, various embodiments described above may be implemented in a recording medium readable by a computer or a similar device using software, hardware, or a combination thereof.

In hardware implementations, the embodiments described in the present disclosure may be implemented using at least one of application specific integrated circuits (ASICs), digital signal processors (DSPs), digital signal processing devices (DSPDs), programmable logic devices (PLDs), field programmable gate arrays (FPGAs), processors, controllers, microcontrollers, microprocessors, and other electrical units for performing functions.

In some cases, the embodiments described herein may be implemented using the processor itself. In software implementations, the embodiments described herein, such as the procedures and functions described herein, may be implemented as separate software modules. Each of the software modules described above may perform one or more functions and operations described herein.

Meanwhile, computer instructions for performing processing operations in electronic devices and the like according to the various embodiments of the present disclosure described above may be stored on a non-transitory computer-readable medium. When executed by a processor of a specific device, the computer instructions stored on the non-transitory computer-readable medium cause the above-described specific device to perform the processing operations described above according to the various embodiments.

The non-transitory computer-readable medium is a medium that can semi-permanently store data and can be read by a device, rather than a medium that stores data for a short period of time, such as a register, cache, or memory. Specific examples of non-transitory computer-readable medium include CDs, DVDs, hard disks, Blu-ray discs, USB memories, memory cards, and ROMs.

While preferred embodiments of the present disclosure have been illustrated and described above, the present disclosure is not limited to the specific embodiments described above, and various modifications may be made by those skilled in the art without departing from the gist of the present disclosure as claimed in the claims and should not be individually understood from the technical spirit or perspective of the present disclosure.

### Description of Reference Numerals

100: Electronic device

110: Memory

120: Processor

130: Communication interface

200: User terminal

300: Body temperature measurement device

What is claimed is:

1. A method of operating an electronic device connected to a user terminal and a non-contact body temperature measurement device, the method comprising:
acquiring variable data including a user’s age and sex through user input received from the user terminal;
setting a user’s normal body temperature range based on data output by inputting the variable data including the user’s age and sex to at least one artificial intelligence model trained to determine the normal body temperature range based on at least one element included in the variable data; and
comparing the user’s body temperature sensed by the body temperature measurement device with the set normal body temperature range to predict the user’s disease.

2. The method of claim 1, comprising:
receiving information on an external environment temperature of the body temperature measurement device from the body temperature measurement device;
acquiring information on at least one physical activity performed by the user through the user input received from the user terminal; and
adding information on the external environment temperature and the body activity to the variable data.

3. The method of claim 1, comprising:
acquiring training data including a plurality of body temperature ranges and at least one variable element matching each of the plurality of body temperature ranges; and
training the artificial intelligence model based on the training data.

4. The method of claim 1, wherein the predicting of the user’s disease includes
identifying the user’s fever pattern including the user’s hourly body temperature deviating from the set normal body temperature range when the user’s body temperature deviates from the set normal body temperature range;
comparing the user’s fever pattern with the fever pattern preset for each disease to acquire a similarity; and
identifying a disease for which the similarity exceeds a threshold as the user’s disease.

5. The method of claim 4, wherein the identifying of the user’s fever pattern includes
identifying at least one fever point in time at which the user’s body temperature begins to deviate from the set normal body temperature range;
calculating, for each fever point in time, a fever period during which the user’s body temperature continuously deviates from the set normal body temperature range;
identifying, for each fever point in time, whether the user’s body temperature increases or decreases; and
identifying the fever pattern including at least one of a time interval between a plurality of fever points in time, a fever period for each of the plurality of fever points in time, and whether the user’s body temperature increases or decreases.

6. The method of claim 5, wherein the comparing of the user’s fever pattern with the fever pattern preset for each disease to acquire the similarity includes acquiring the similarity of the user’s fever pattern to the fever pattern preset for each disease based on data output by inputting the user’s fever pattern and the fever pattern preset for each disease to at least one similarity calculation model trained to calculate similarity between data based on data embedding.

7. The method of claim 1, comprising:
identifying a body temperature pattern related to the user’s body temperature change cycle, based on a body temperature measurement history including the user’s hourly body temperature divided by day; and
correcting the user’s normal body temperature range based on the identified body temperature pattern.

8. The method of claim 7, wherein the identifying of the body temperature pattern includes
identifying at least one rising time period in which the user’s body temperature rises daily and identifying at least one first time period in which a plurality of ones of the daily identified rising time periods overlap;
identifying at least one falling time period in which the user’s body temperature falls daily and identifying at least one second time period in which a plurality of ones of the daily identified falling time periods overlap; and
identifying the user’s body temperature pattern including the first time period and the second time period.

9. The method of claim 8, wherein the correcting of the user’s normal body temperature range includes
calculating a first average value, which is an average value of the body temperature identified during the first time period, and a second average value, which is an average value of body temperature identified during the second time period, based on the body temperature measurement history;
correcting the user’s normal body temperature range based on the first average value to acquire a first body temperature range;
setting the first body temperature range to the user’s normal body temperature range during the first time period;
correcting the user’s normal body temperature range based on the second average value to acquire a second body temperature range; and
setting the second body temperature range as the user’s normal body temperature range during the second time period.
